(19) United States
(12) Patent Application Publication
(10) Pub. No.: US 2022/0192562 A1
(43) Pub. Date: Jun. 23, 2022

(54) ARTIFICIAL INTELLIGENCE INTENTION AND BEHAVIOR FEEDBACK METHOD, APPARATUS AND SYSTEM

(71) Applicant: THE COLLECTIVE INTENTIONS LIMITED, Taunton (GB)

(72) Inventors: James S. HAYES, Greer, SC (US); David C. ATKINSON, Taunton (GB)

(73) Assignee: THE COLLECTIVE INTENTIONS LIMITED, Taunton (GB)

(21) Appl. No.: 17/561,295

(22) Filed: Dec. 23, 2021

Related U.S. Application Data
(60) Provisional application No. 63/130,264, filed on Dec. 23, 2020.

(51) Int. Cl. A61B 5/16; G06N 3/08

(52) U.S. Cl. CPC A61B 5/167; G06N 3/08

(57) ABSTRACT

Methods, apparatus, and system for an artificial intelligence intention and behavior feedback module to receive and organize intentions, goals, tasks, behaviors, and personality types for a large group of individuals and to provide feedback to an individual regarding the tasks, behaviors, and personality type of the individual, to predict behaviors, to predict outcomes of predicted behaviors, to suggest alternative behaviors, to suggest alternative tasks, and to suggest mentorship relationships among the large group of individuals.

### FIELD

The present disclosure relates to a computing device, in particular to, a computing device to use artificial intelligence, such as neural networks, to analyze intentions and behavior.

### BACKGROUND

Humans often think of themselves as being a unitary person, with one brain and nervous system which determines or controls the person's intentions and goals, the tasks we take on to achieve the intentions and goals, and how we act out tasks through behavior. However, human (and non-human) brain and nervous systems may be more complex than this.

The brain and nervous system may comprise multiple interacting feedback processes. These multiple interacting feedback processes may have different intentions or goals, may operate at different time-scales, may be dominant in response to different input or stimuli, may be conscious and deliberate or may be reflexive, performed by the basal ganglia in a “reptilian complex” or “lizard brain,” and may alternate which process is dominant in the brain's metabolism at any given time.

Individuals may not have an objective view of these multiple interacting feedback processes and may not understand or appreciate how they may be synergistic, antagonistic, or neutral.

The multiple interacting feedback processes in individuals may be influenced by trained and or biologically determined biases which may influence control by and or timing of the multiple interacting feedback processes. The trained and or biologically determined biases may be organized into personality types.

An Enneagram of Personality is an example of a personality type matrix. The Enneagram of Personality defines nine personality types, wherein the types are represented as points of a geometric figure referred to as an enneagram, with connections between the points or types. Other arrangements of personality types, as well as connections between the types, are known and may be developed in the future.

A person may benefit from observing multiple interacting feedback processes within themself, from learning where or how such person may be categorized in an enneagram or personality type matrix, and from interactions with others who may also be categorized in a personality type matrix, though existing approaches to such topics, such as psychoanalysis, therapy, and the like, may be expensive, time consuming, not desirable and or not available if desired.

Therefore, there is a need for a system, method, and apparatus to programmatically observe an individual, identify multiple interacting feedback processes within such person, to learn where or how such person may be categorized in a personality type matrix, such as an enneagram, to communicate such information to the person, and to facilitate interactions by the person with others who have gone through similar situations, more or less successfully, with a similar personality type.

### BRIEF DESCRIPTION OF THE DRAWINGS

FIG. 1 is a network and device diagram illustrating an example of an individual, an organization, a behavior computer device, an intention computer device, a user interface computer device, an intention and behavior feedback computer device, an intention and behavior feedback computer device datastore, and a network incorporated with teachings of the present disclosure, according to some embodiments.

FIG. 2 is a functional block diagram illustrating an example of the intention and behavior feedback computer device of FIG. 1, incorporated with teachings of the present disclosure, according to some embodiments.

FIG. 3 is a functional block diagram illustrating an example of an intention and behavior feedback computer device datastore incorporated with teachings of the present disclosure, consistent with embodiments of the present disclosure.

FIG. 4 is a flow diagram illustrating an example of a method performed by an intention and behavior feedback module, according to some embodiments.

FIG. 5 is a flow diagram illustrating an example of a method performed by a method module, according to some embodiments.

FIG. 6 is a flow diagram illustrating an example of a method performed by a behavior module, according to some embodiments.

FIG. 7 is a flow diagram illustrating an example of a method performed by a relation module, according to some embodiments.

FIG. 8 is a flow diagram illustrating an example of a method performed by an intention and behavior integration module, according to some embodiments.

FIG. 9 illustrates an example of a user interface, illustrating a current task and evaluation thereof, according to some embodiments.

FIG. 10 illustrates an example of a user interface, providing analysis of personality traits, according to some embodiments.

FIG. 11 is an example of a user interface, providing a view of intentions, goals, current task(s), current behavior(s), predicted behavior(s), alternative behavior(s), and alternative tasks personality traits, according to some embodiments.

FIG. 12 illustrates an example of a first system architecture, according to some embodiments.

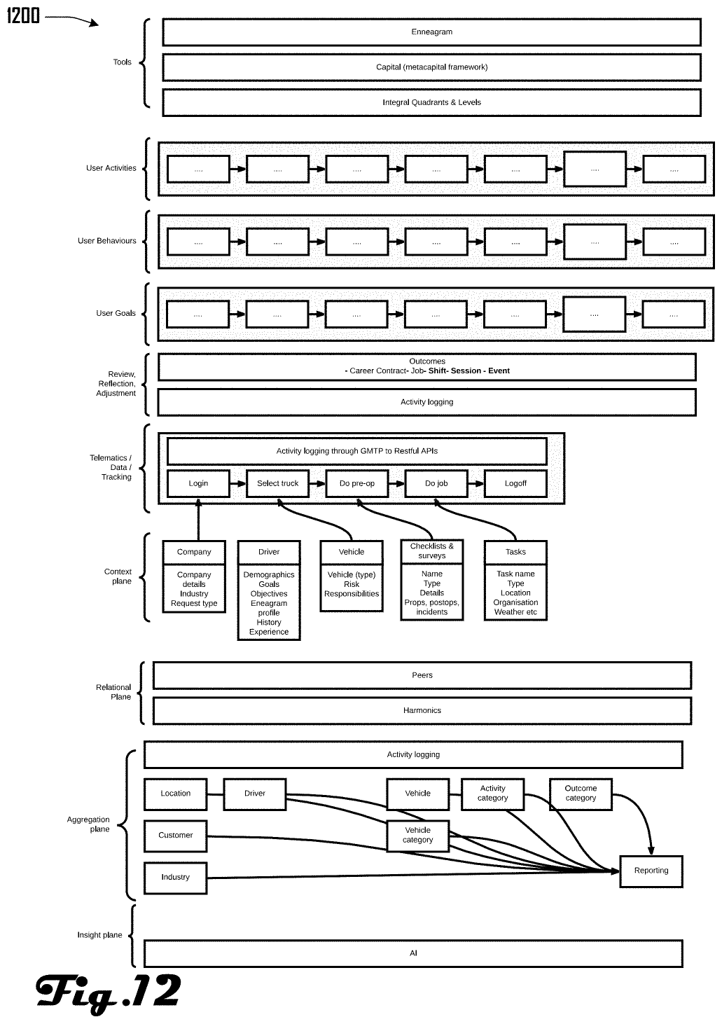

FIG. 13 illustrates an example of a second system architecture, according to some embodiments.

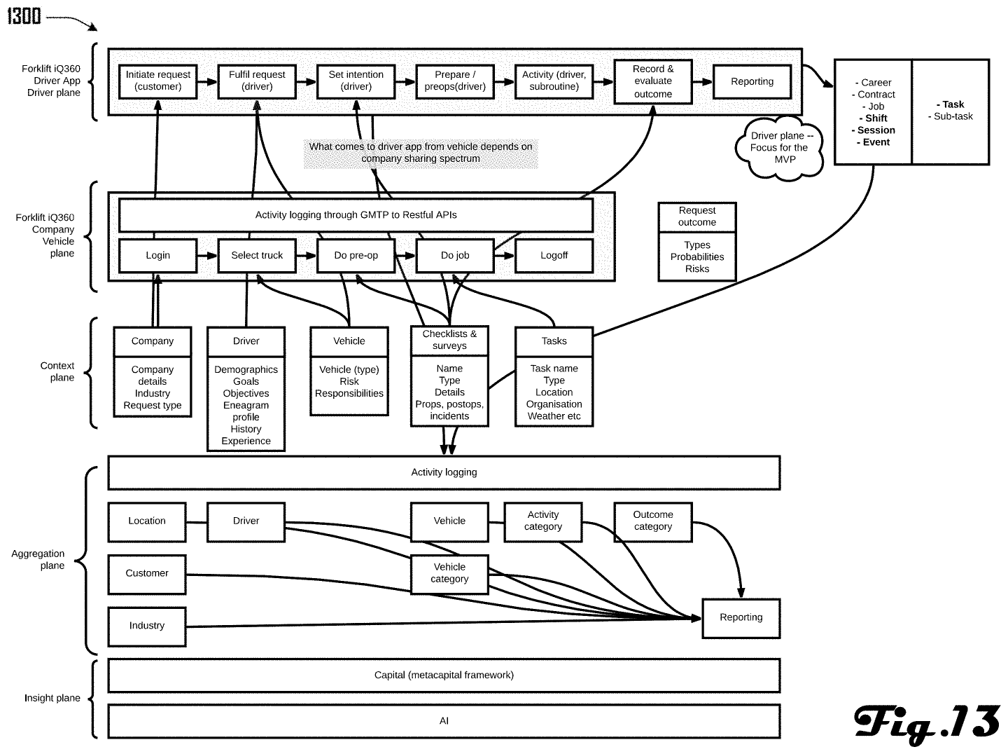

### DETAILED DESCRIPTION

In addition to other locations, defined terms may be found at the end of this Detailed Description.

In overview, this disclosure relates to an apparatus and methods performed by and in an intention and behavior feedback computer device apparatus or system to programmatically observe and interact with an individual, identify multiple interacting feedback processes within such person, to prepare and update matrices of intentions, goals, tasks, behaviors, e.g. in a personality type matrix, e.g. an enneagram structure, to determine how such person may be categorized according to the personality type matrix, to communicate such information to the person, and to facilitate interactions by the person with others who have gone through similar situations and who have a similar personality type. The system may comprise one or more computer devices. The system may facilitate interaction between an individual and an organization, such as an employer.

The intention and behavior feedback computer device apparatus may include a hardware acceleration module to accelerate the performance of the modules by hardware of the intention and behavior feedback computer device, for example, to perform neural network processes and allow other modules to operate in what a user perceives as real time.

In this way, the intention and behavior feedback computer device apparatus may programmatically observe and interact with an individual, identify multiple interacting feedback processes within such person, prepare and update matrices of intentions, goals, tasks, behaviors, e.g. in or in relation to a personality type matrix, e.g. an enneagram structure, determine how such person may be categorized according to the personality type matrix, communicate such information to the person, and facilitate interactions by the person with others who have gone through similar situations and who have a similar personality type.

The services provided by intention and behavior feedback computer device apparatus are provided in part through neural network analysis of data from a large group of people. The neural network analysis effectively graphs, in a tractable manner, intentions, objectives, tasks, behaviors, and outcomes, in addition to personality types. The neural network is also used to analyze an individual and to provide the individual with neural network analysis, to allow the individual to see the individual's behavior, to predict what outcomes will come from the behavior, to predict behaviors of the individual, to predict what may outcomes may come from predicted behaviors, and to suggest alternative behaviors and tasks to achieve intentions and goas, in view of the individual's personality type.

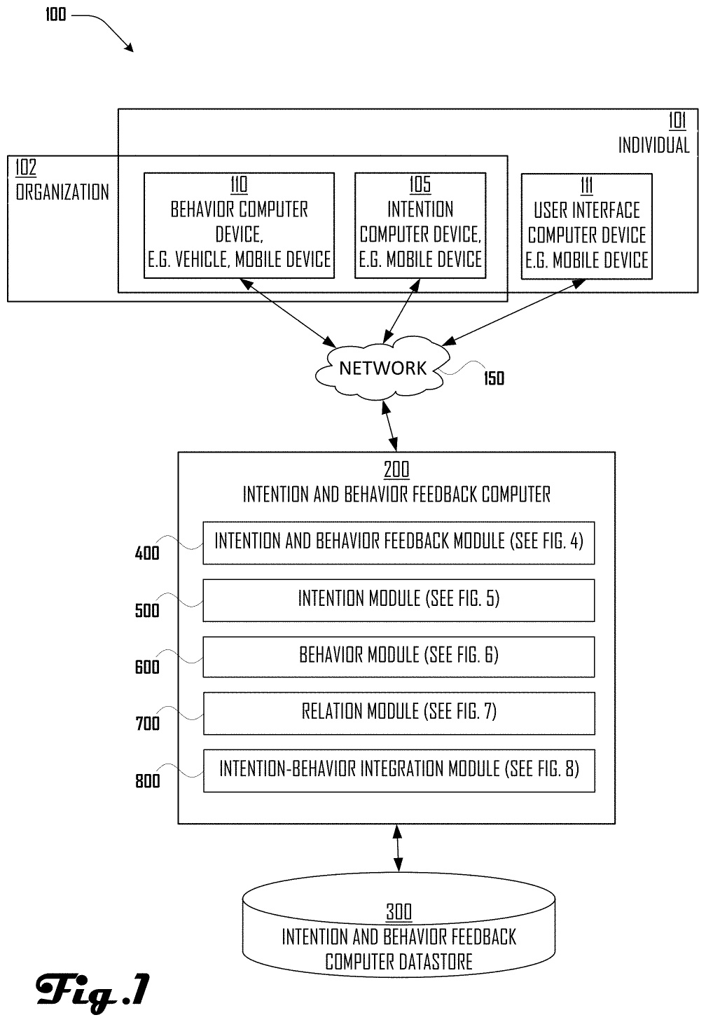

FIG. 1 is a network and device diagram illustrating an example of individual 101, organization 102, behavior computer device 110, intention computer device 105, user interface computer device 111, intention and behavior feedback computer device 200, intention and behavior feedback computer device datastore 300, and network 150 incorporated with teachings of the present disclosure, according to some embodiments.

Individual 101 may be a human person or individual. Individual 101 may possess or have access to user interface computer device 111, e.g. a mobile computer device, such as a mobile phone. Individual 101 may further possess or have access to intention computer device 105, e.g. a mobile computer device, such as a mobile phone. User interface computer device 111 and intention computer device 105 may be a same mobile computer device and or different computer devices. Individual 101 may further possess or have access to behavior computer device 110, e.g. a mobile computer device, such as a mobile phone, a vehicle, a work computer, or the like.

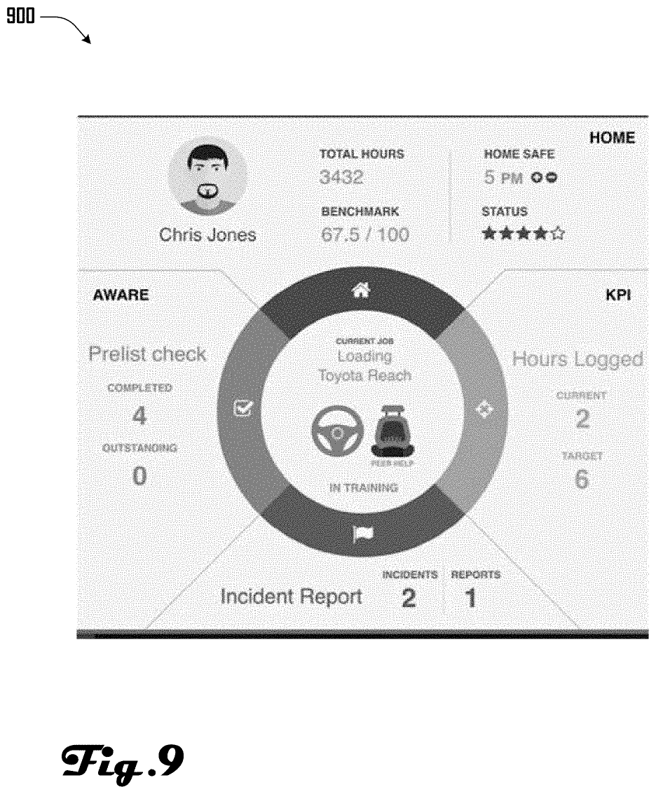

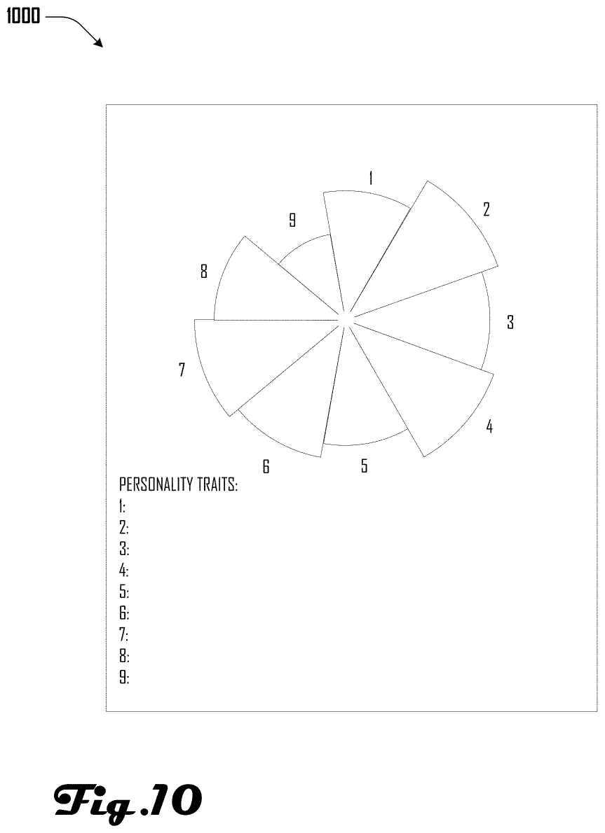

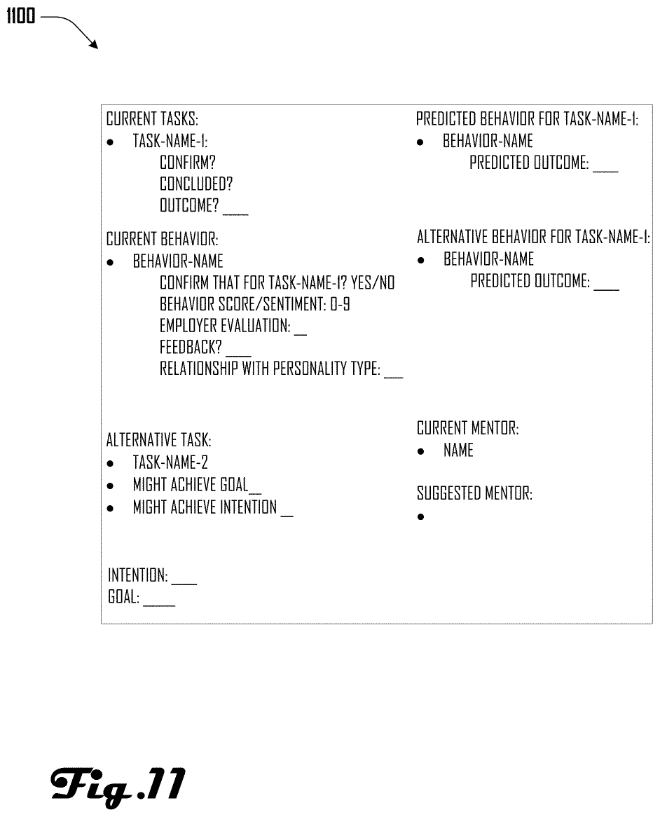

User interface computer device 111 may perform executable software to enable a user interface to the modules described herein. Examples of one or more screens, windows, or the like, of user interfaces or components thereof are illustrated in relation to user interface 900 in FIG. 9, user interface 1000 in FIG. 10, and user interface 1100 in FIG. 11.

Organization 102 may be an entity, such as an employer, a corporation, a nonprofit organization, or the like. Organization 102 and or individual 101 may have a relationship, such as an employer-employee relationship or the like. Organization 102 and or individual 101 may provide one or more of intention computer device 105 and behavior computer device 110.

Intention computer device 105 and or behavior computer device 110 may perform intention and behavior feedback module 400.

Intention and behavior feedback module 400 may call intention module 500 and perform intention module 500 in conjunction with intention computer device 105. Intention module 500 may obtain information regarding an individual, organizations and other individuals related to the individual, obtain and update user intentions and goals, obtain tasks of the individual, and form or update a matrix of intentions, goals, tasks of the individual, e.g. in an enneagram of the individual.

Intention and behavior feedback module 400 may call behavior module 600 and perform behavior module 600 in conjunction with behavior computer device 110. Behavior module 600 may determine a behavior collection device 110 relative to the individual, obtain tasks performed by the individual with behavior collection device 110, obtain user behaviors with, toward, through behavior collection device 110 toward tasks and otherwise, and form or update a matrix of behaviors of the individual, e.g. in an enneagram of the individual.

Intention and behavior feedback module 400 may call and perform relation module 700 to identify peers; peers are other individuals; peers have a range of similarity, difference, or distance relative to an individual. Relation module 700 may identify and update communication connections or edges between nodes of peers (“peer edges”), relation module 700 may perform a neural network to identify peer harmonics within the nodes and edges, including synergistic, antagonistic, and neutral communication harmonics among peers. Relation module 700 may update a personality type matrix of the individual with the connections or edges; e.g. In the enneagram of the individual.

Intention and behavior feedback module 400 may call and perform intention-behavior integration module 800 to train an artificial intelligence model, e.g. a plurality of neural networks in, for example, a transformer architecture, on or with respect to matrices of intentions, goals, tasks, behaviors, peer edges, and personality types, e.g. on enneagrams, for a large group of individuals. The artificial intelligence model may then be used with respect to an individual to identify the individual's personality type, to identify others who have most similar goals and personality types, to identify tasks and behaviors of others which were closer to or further from goals, to identify blocks, to suggest tasks and behaviors to achieve goals, and to suggest communication with others.

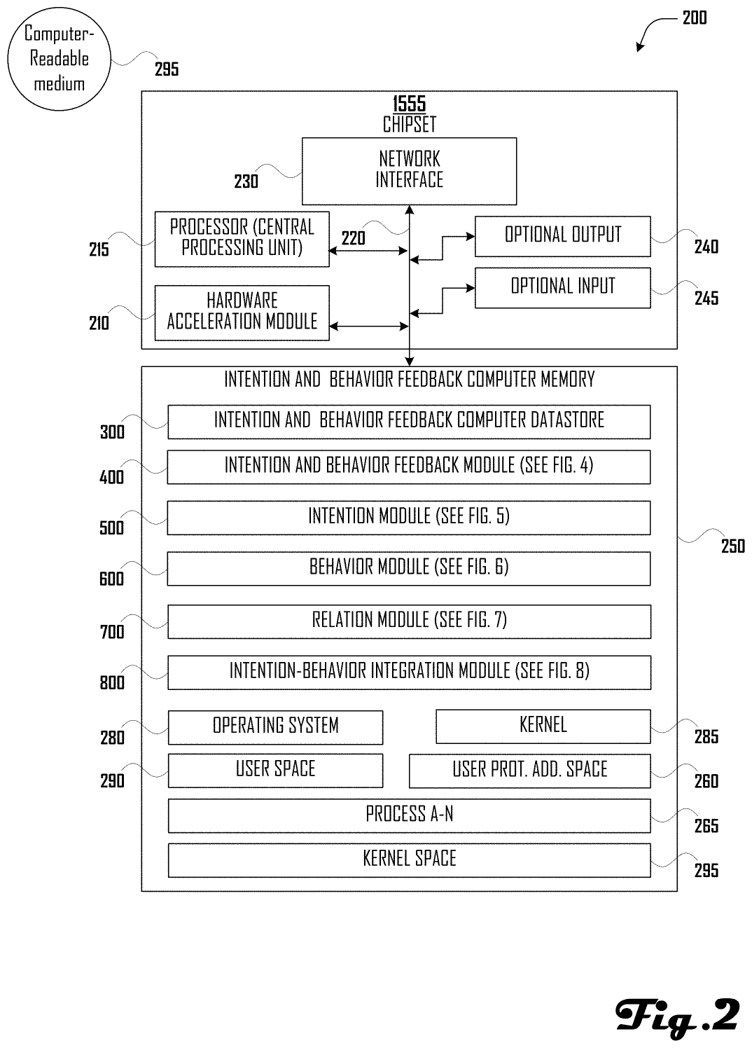

Behavior computer device 110, intention computer device 105, and or user interface computer device 111 illustrated in FIG. 1 may be connected with network 150 and/or intention and behavior feedback computer 200, described further in relation to FIG. 2.

Intention and behavior feedback computer 200 is illustrated as connecting to intention and behavior feedback computer datastore 300. Intention and behavior feedback computer datastore 300 is described further, herein, though, generally, should be understood as a datastore used by intention and behavior feedback computer 200.

Network 150 may comprise computers, network connections among the computers, and software routines to enable communication between the computers over the network connections. Examples of Network 150 comprise an Ethernet network, the Internet, and/or a wireless network, such as a GSM, TDMA, CDMA, EDGE, HSPA, LTE or other network provided by a wireless service provider. Connection to Network 150 may be via a Wi-Fi connection. More than one network may be involved in a communication session between the illustrated devices. Connection to Network 150 may require that the computers execute software routines which enable, for example, the seven layers of the OSI model of computer networking or equivalent in a wireless phone network.

FIG. 2 is a functional block diagram illustrating an example of intention and behavior feedback computer 200, incorporated with teachings of the present disclosure, according to some embodiments. Intention and behavior feedback computer 200 may include chipset 255. Chipset 255 may include processor 215, input/output (I/O) port(s) and peripheral devices, such as output 240 and input 245, and network interface 230, and computer device memory 250, all interconnected via bus 220. Network interface 230 may be utilized to form connections with network 150, with intention and behavior feedback computer datastore 300, or to form device-to-device connections with other computers.

Chipset 255 may include communication components and/or paths, e.g., buses 220, that couple processor 215 to peripheral devices, such as, for example, output 240 and input 245, which may be connected via I/O ports. Processor 215 may include one or more execution cores (CPUs). For example, chipset 255 may also include a peripheral controller hub (PCH) (not shown). In another example, chipset 255 may also include a sensors hub (not shown). Input 245 and output 240 may include, for example, user interface device(s) including a display, a touch-screen display, printer, keypad, keyboard, etc., sensor(s) including accelerometer, global positioning system (GPS), gyroscope, etc., communication logic, wired and/or wireless, storage device(s) including hard disk drives, solid-state drives, removable storage media, etc. I/O ports for input 245 and output 240 may be configured to transmit and/or receive commands and/or data according to one or more communications protocols. For example, one or more of the I/O ports may comply and/or be compatible with a universal serial bus (USB) protocol, peripheral component interconnect (PCI) protocol (e.g., PCI express (PCIe)), or the like.

Hardware acceleration module 210 may provide hardware acceleration of various functions otherwise performed by intention and behavior feedback module 400, intention module 500, behavior module 600, relation module 700, and/or intention-behavior integration module 800. Hardware acceleration module may be provided by, for example, Integrated Performance Primitives software library by Intel Corporation, as may be executed by an Intel (or other compatible) chip, and which may implement, for example, a library of programming functions involved with real time computer vision and machine learning systems. Such a library includes, for example, OpenCV. OpenCV includes, for example, application areas including 2D and 3D feature toolkits, egomotion estimation, facial recognition, gesture recognition, human-computer interaction, mobile robotics, motion understanding, object identification, segmentation and recognition, stereopsis stereo vision (including depth perception from two cameras), structure from motion, motion tracking, and augmented reality. OpenCV also includes a statistical machine learning library including boosting, decision tree learning, gradient boosting trees, expectation-maximization algorithms, k-nearest neighbor algorithm, naïve Bayes classifier, artificial neural networks, random forest, and a support vector machine.

Hardware acceleration module may be provided by, for example, NVIDIA® CUDA-X libraries, tools, and technologies built on NVIDIA CUDA® technologies. Such libraries may comprise, for example, math libraries, parallel algorithms, image and video libraries, communication libraries, deep learning libraries, and partner libraries. Math libraries may comprise, for example, a GPU-accelerated basic linear algebra (BLAS) library, a GPU-accelerated library for Fast Fourier Transforms, a GPU-accelerated standard mathematical function library, a GPU-accelerated random number generation (RNG), GPU-accelerated dense and sparse direct solvers, GPU-accelerated BLAS for sparse matrices, a GPU-accelerated tensor linear algebra library, and a GPU-accelerated linear solvers for simulations and implicit unstructured methods. Parallel algorithm libraries may comprise, for example a GPU-accelerated library of C++ parallel algorithms and data structures. Image and video libraries may comprise, for example, a GPU-accelerated library for JPEG decoding, GPU-accelerated image, video, and signal processing functions, a set of APIs, samples, and documentation for hardware accelerated video encode and decode on various operating systems, and a software developer kit which exposes hardware capability of NVIDIA TURING™ GPUs dedicated to computing relative motion of pixels between images. Communication libraries may comprise a standard for GPU memory, with extensions for improved performance on GPUs, an open-source library for fast multi-GPU, multi-node communications that maximize bandwidth while maintaining low latency. Deep learning libraries may comprise, for example, a GPU-accelerated library of primitives for deep neural networks, a deep learning inference optimizer and runtime for product deployment, a real-time streaming analytics toolkit for AI-based video understanding and multi-sensor processing, and an open source library for decoding and augmenting images and videos to accelerate deep learning applications. Partner libraries may comprise, for example, OpenCV, FFmpeg, ArrayFire, Magma, IMSL Fortan Numerical Library, Gunrock, Cholmod, Triton Ocean SDK, CUVIIib, and others.

In embodiments, hardware acceleration module 210 may be or comprise a programmed FPGA, i.e., a FPGA which gate arrays are configured with a bit stream to embody the logic of the hardware accelerated function (equivalent to the logic provided by the executable instructions of a software embodiment of the function). In embodiments, hardware acceleration module 210 may also or alternatively include components of or supporting computer device memory 250.

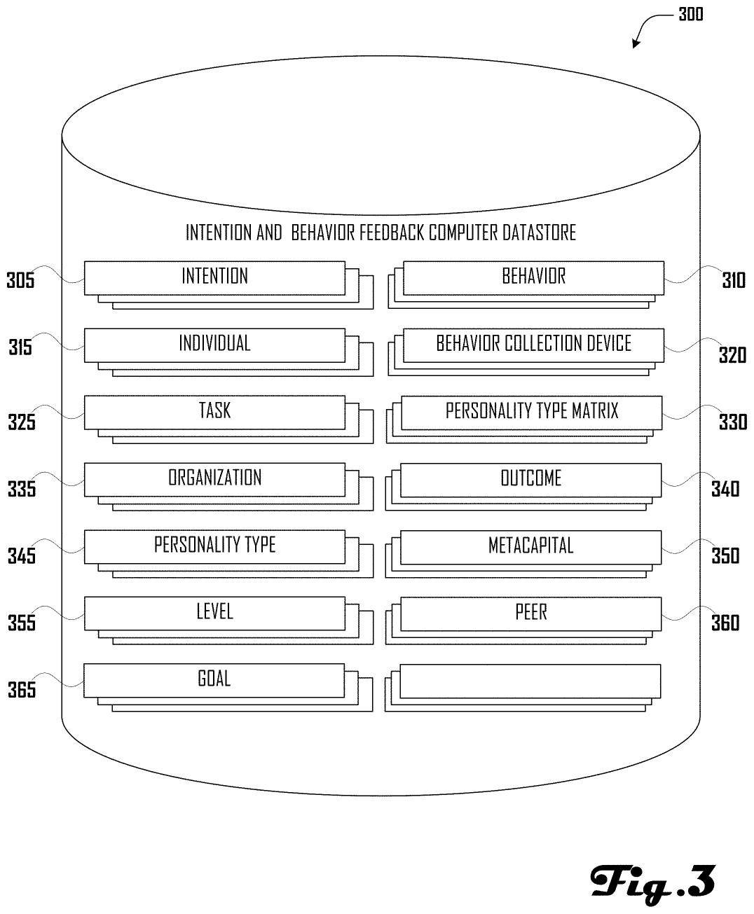

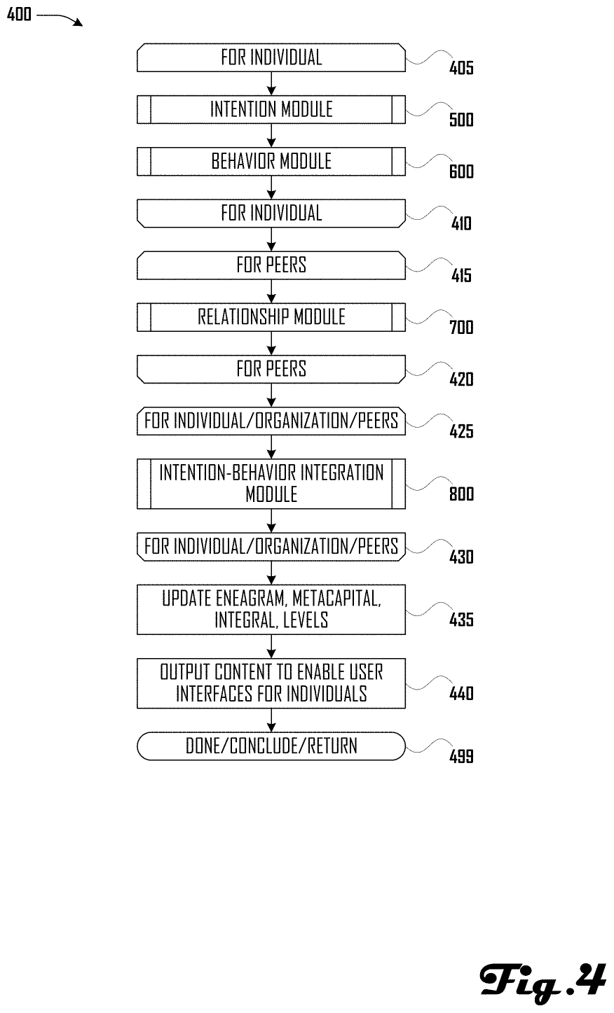

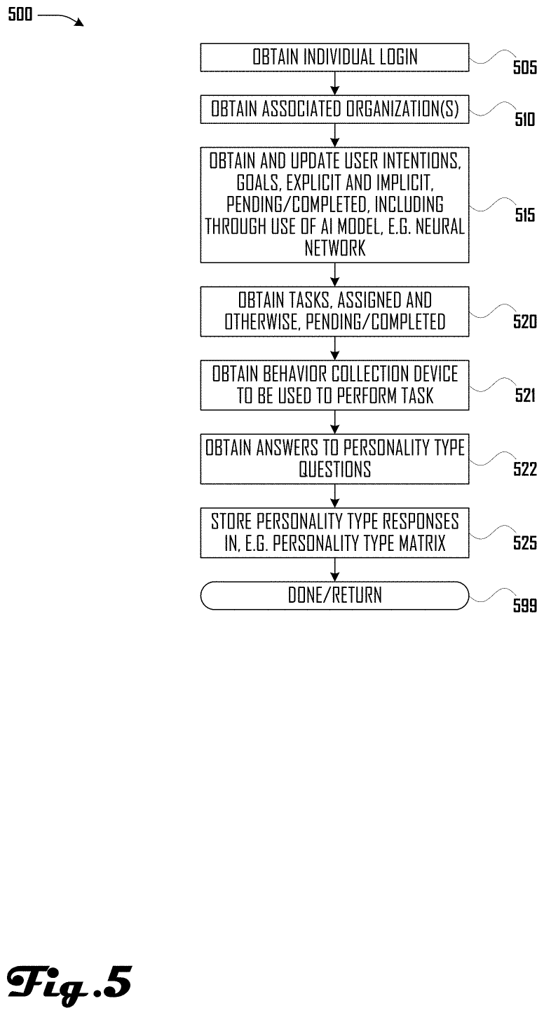

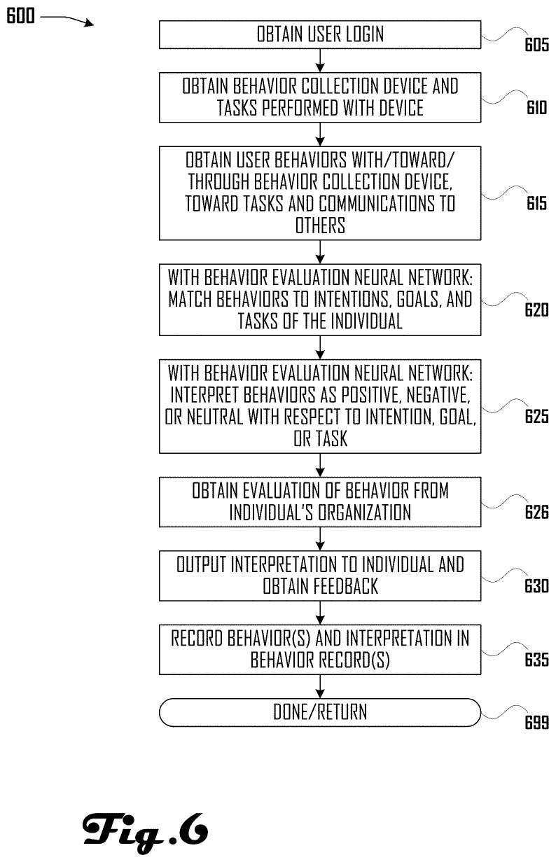

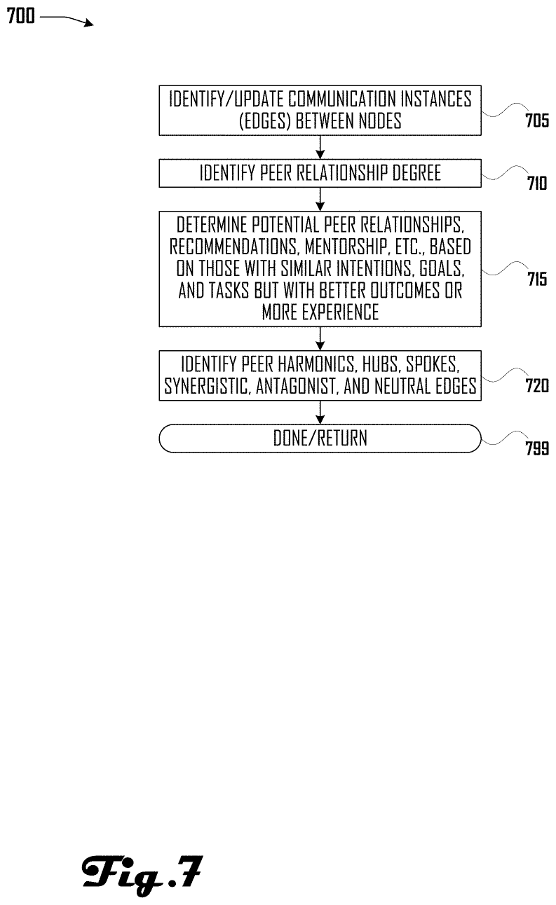

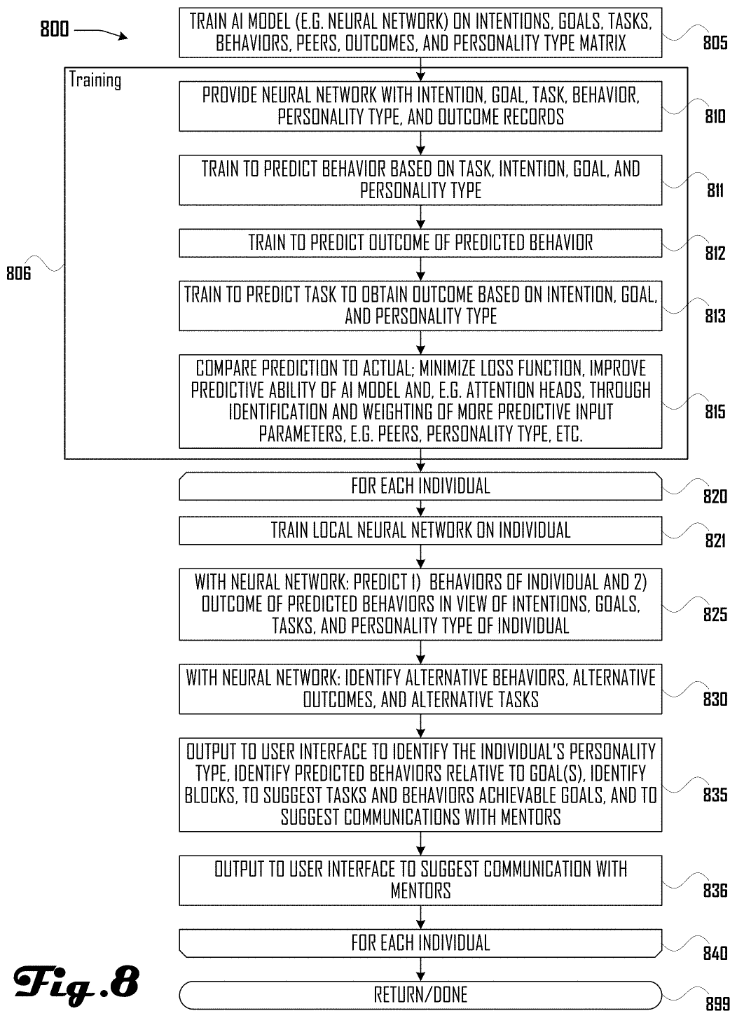

Computer device memory 250 may generally comprise a random access memory (“RAM”), a read only memory (“ROM”), and a permanent mass storage device, such as a disk drive or SDRAM (synchronous dynamic random-access memory). Computer device memory 250 may store program code for modules and/or software routines, such as, for example, hardware acceleration module 210, intention and behavior feedback computer datastore 300 (illustrated and discussed further in relation to FIG. 3), intention and behavior feedback module 400 (illustrated and discussed further in relation to FIG. 4), intention module 500 (illustrated and discussed further in relation to FIG. 5), behavior module 600 (illustrated and discussed further in relation to FIG. 6), relation module 700 (illustrated and discussed further in relation to FIG. 7), and intention-behavior integration module 800 (illustrated and discussed further in relation to FIG. 8).

Computer device memory 250 may also store operating system 280. These software components may be loaded from a non-transient computer readable storage medium 295 into computer device memory 250 using a drive mechanism associated with a non-transient computer readable storage medium 295, such as a floppy disc, tape, DVD/CD-ROM drive, memory card, or other like storage medium. In some embodiments, software components may also or instead be loaded via a mechanism other than a drive mechanism and computer readable storage medium 295 (e.g., via network interface 230).

Computer device memory 250 is also illustrated as comprising kernel 285, kernel space 295, user space 290, user protected address space 260, and intention and behavior feedback computer datastore 300 (illustrated and discussed further in relation to FIG. 3).

Computer device memory 250 may store one or more process 265 (i.e., executing software application(s)). Process 265 may be stored in user space 290. Process 265 may include one or more other process 265a . . . 265n. One or more process 265 may execute generally in parallel, i.e., as a plurality of processes and/or a plurality of threads.

Computer device memory 250 is further illustrated as storing operating system 280 and/or kernel 285. The operating system 280 and/or kernel 285 may be stored in kernel space 295. In some embodiments, operating system 280 may include kernel 285. Operating system 280 and/or kernel 285 may attempt to protect kernel space 295 and prevent access by certain of process 265a . . . 265n.

Kernel 285 may be configured to provide an interface between user processes and circuitry associated with intention and behavior feedback computer 200. In other words, kernel 285 may be configured to manage access to processor 215, chipset 255, I/O ports and peripheral devices by process 265. Kernel 285 may include one or more drivers configured to manage and/or communicate with elements of intention and behavior feedback computer 200 (i.e., processor 215, chipset 255, I/O ports and peripheral devices).

Intention and behavior feedback computer 200 may also comprise or communicate via Bus 220 and/or network interface 230 with intention and behavior feedback computer datastore 300, illustrated and discussed further in relation to FIG. 3. In various embodiments, bus 220 may comprise a high-speed serial bus, and network interface 230 may be coupled to a storage area network (“SAN”), a high speed wired or wireless network, and/or via other suitable communication technology. intention and behavior feedback computer 200 may, in some embodiments, include many more components than as illustrated. However, it is not necessary that all components be shown in order to disclose an illustrative embodiment.

FIG. 3 is a functional block diagram of the intention and behavior feedback computer datastore 300 illustrated in the computer device of FIG. 2, according to some embodiments. The components of intention and behavior feedback computer datastore 300 may include data groups used by modules and/or routines, e.g., intention 305, behavior 310, individual 315, behavior collection device 320, task 325, personality type matrix 330, organization 335, outcome 340, personality type 345, metacapital 350, level 355, and peer 360 (to be described more fully below). The data groups used by modules or routines illustrated in FIG. 3 may be represented by a cell in a column or a value separated from other values in a defined structure in a digital document or file. Though referred to herein as individual records or entries, the records may comprise more than one database entry. The database entries may be, represent, or encode numbers, numerical operators, binary values, logical values, text, string operators, references to other database entries, joins, conditional logic, tests, and similar.

The components of computer datastore 300 are discussed further herein in discussion of other of the Figures.

FIG. 4 is a flow diagram illustrating an example of intention and behavior feedback module 400 which may be performed by intention and behavior feedback computer device(s), such as intention and behavior feedback computer 200, according to some embodiments. These modules may be performed by or with the assistance of a hardware accelerator, such as hardware acceleration module 210.

Opening loop block 405 to closing loop block 410 may iterate over an individual, such as an individual 315 record.

At block 500, intention and behavior feedback module 400 may call intention module 500, described further herein. In overview, intention module 500 may obtain organizations associated with an individual, may obtain an individual's intentions and goals, may obtain tasks assigned or assumed by the individual, may obtain a behavior collection device 110 to be used by the individual to perform tasks, may obtain answers to personality type questions, and may store the result thereof in a personality type matrix.

At block 600, intention and behavior feedback module 400 may call behavior module 600, described further herein. In overview, behavior module 600 may obtain behaviors performed by the individual with the behavior collection device 110 toward tasks and in communications to other, may perform a neural network to match behaviors to intentions, goals, and tasks of the individual, may perform a neural network to interpret the behaviors as positive, negative, or neutral with respect to an intention, goal, or task, may obtain an organization's evaluation of the individual's behavior toward a task, may record such information, and may output such information to the individual.

Opening loop block 415 to closing loop block 420 may iterate over peers. Peers may constitute all individual 315 records accessible to intention and behavior feedback module 400 or a subset of individuals accessible to intention and behavior feedback module 400 who have similarities above a threshold.

At block 700, intention and behavior feedback module 400 may call relationship module 700, described further herein. In overview, relationship module 700 may identify communication instances between nodes, may determine a peer relationship degree, may determine potential peer relationships, e.g. mentorship, with those who have similar intentions, goals, and tasks, but who may have better outcomes or more experience, and may identify peer harmonics among peers.

Opening loop block 425 to closing loop block 430 may iterate over, for example, individual 315, organization 335, and or peer 360 records.

At block 800, intention and behavior feedback module 400 may call intention-behavior integration module 800, described further herein. In overview, intention-behavior integration module 800 may train a neural network on intention, goal, task, behavior, personality type, and outcome for a large group of individuals, may train to predict behavior based thereon, may train to predict outcome of predicted behavior, may train to predict a task to obtain an outcome, may train a local neural network on a specific individual regarding the foregoing, and may use the neural network to predict behavior of the individual, outcome of predicted behavior, to provide alternative behaviors and alternative tasks to obtain goals and intentions of the individual, and may output the foregoing to the individual.

At block 435, intention and behavior feedback module 400 may update matrices of intentions, goals, tasks, behaviors, and or personality type, such as in personality type matrix 330 records, for individual 315 records in the system. At block 435, intention and behavior feedback module 400 may further update metacapital 350 records and or level 355 records.

At block 440, intention and behavior feedback module 400 may output content to enable user interfaces for individual 315 records in the system. Examples of user interfaces are illustrated and discussed in relation to FIG. 9, and FIG. 10. In overview,

At block 499, intention and behavior feedback module 400 may conclude and/or return to a module and/or another process which may have called it.

FIG. 5 is a flow diagram illustrating an example of intention module 500 which may be performed by intention and behavior feedback computer device(s), such as intention and behavior feedback computer 200, according to some embodiments. These modules may be performed by or with the assistance of a hardware accelerator, such as hardware acceleration module 210. This module may be performed with data and or assistance from, for example, intention computer device 105.

At block 505, intention module 500 may obtain a login or other identifier for individual 315.

At block 510, intention module 500 may obtain organizations and other individuals associated with the then-current individual 315 record, such as employers, organizations in which the individual works or participates, family, co-workers, and the like. These may be obtained from the individual; these may be obtained by mining records for likely connections and confirming the associations with the individual.

At block 515, intention module 500 may obtain and update user intentions and goals. These may be obtained by interaction with and input from the individual, e.g. through user interface computer device 111. These may be obtained by intention module 500 executing a neural network to interpret communications of individual with others through or with user interface computer device 111, with intention computer device 105, with behavior computer device 110, and the like, as communicating intentions and goals, whether explicit or implicit. The neural network may be trained on communication data, labeled with respect to intentions and goals. Interpreted intentions and goals may be confirmed with the individual. Intentions and goals may be stored as, for example, one or more intention 305 record and one or more goal 365 record. At block 515, intention module 500 may determine or obtain whether intentions and goals are pending or completed or a distance from an intention or a goal.

At block 520, intention module 500 may obtain tasks of the individual. Tasks may be obtained from, for example, the individual, an organization associated with the individual, and the like. Tasks may be assigned to the individual, according to the individual's role in an organization. Tasks may be obtained by interaction with the individual, from communications of individual with others through or with user interface computer device 111, with intention computer device 105, with behavior computer device 110, and the like. Tasks may be stored as, for example, one or more task 325 record. At block 520, intention module 500 may further determine or obtain whether tasks are pending or completed.

At block 521, intention module 500 may obtain or identify a behavior collection device to be used to perform a task.

At block 522, intention module 500 may query the individual regarding and may obtain answers to personality type questions, e.g. to questions design to categorize the individual in a personality type matrix, e.g. in an enneagram.

At block 525, intention module 500 may store responses to personality queries of block 522 in, for example, one or more personality type matrix 330 record.

At block 599, intention module 500 may conclude and/or return to a module and/or another process which may have called it.

FIG. 6 is a flow diagram illustrating an example of behavior module 600 which may be performed by intention and behavior feedback computer device(s), such as intention and behavior feedback computer 200, according to some embodiments. These modules may be performed by or with the assistance of a hardware accelerator, such as hardware acceleration module 210. This module may be performed with data and or assistance from, for example, behavior computer device 110.

At block 605, behavior module 600 may obtain a login or other identifier for individual 315.

At block 610, behavior module 600 may obtain behavior collection device(s) associated with the individual, such as one or more behavior computer device 110. For example, if the individual is assigned to use a vehicle by an employer, the vehicle may be a behavior collection device. At block 610, behavior module 600 may further obtain tasks performed with the behavior collection device. These may be obtained by interaction with the individual, user interface computer device 111 in the example user interface 1100 illustrated in FIG. 11, from communications of individual with others through or with user interface computer device 111, with intention computer device 105, with behavior computer device 110, and the like. Behavior collection devices may be stored as, for example, one or more behavior collection device 320 record. One or more associated task 325 records may be updated.

At block 615, behavior module 600 may obtain user behaviors with, toward, or through the behavior collection device, tasks associated with the behaviors, and communications with others. Behaviors may comprise, for example, use of the behavior collection device, such as starting and stopping, navigating, accelerating, decelerating, and other behaviors recorded in sensor information of the behavior collection device, including text, audio, video, and the like, generated by one individual. Association between a behavior and a task may be provided explicitly, by the user, such as by a user indication that a task is being performed with the behavior collection device, e.g. as in the example illustrated in user interface 1100 in FIG. 11. Association between a behavior and a task may be determined by, for example, correlation between a calendar or schedule of tasks, a place or location of a task, and the time and location which the behavior occurred, Behaviors may further comprise communications with others. The behavior collection device may collect who was contacted, when, how, as well as the substance of the contact. The substance of the contact may comprise, for example, word in text, audio, video, and the like.

At block 620, behavior module 600 may execute a behavior evaluation neural network to match behaviors and communications to intentions, goals, and tasks of the individual. This match may be supplemental to information determined in block 615. This match may be presented to an individual for confirmation in a user interface, as illustrated in the example illustrated in user interface 1100 in FIG. 11.

At block 625, behavior module 600 may execute the behavior evaluation neural network to interpret whether the behaviors are positive, negative, or neutral and or a sentiment with respect to an intention or goal associated with the task or person. The neural network may be part of and or executed by the hardware acceleration module. The neural network may be trained on labeled data obtained from the same or other individuals.

At block 626, behavior module 600 may obtain feedback regarding a behavior from, for example, an employer or other organization of the individual.

At block 630, behavior module 600 may output the interpretation of block 625 and the feedback from the organization of individual to the individual and obtain feedback therefrom.

At block 618, behavior module 600 may record the behaviors and interpretations thereof in one or more behavior 310 records.

At block 699, behavior module 600 may conclude and/or return to a module and/or another process which may have called it.

FIG. 7 is a flow diagram illustrating an example of relationship module 700 which may be performed by intention and behavior feedback computer device(s), such as intention and behavior feedback computer 200, according to some embodiments. These modules may be performed by or with the assistance of a hardware accelerator, such as hardware acceleration module 210. This module may be performed with data and or assistance from, for example, behavior computer device 110 and or user interface computer device 111.

At block 705, relationship module 700 may identify and update communication connections or edges between nodes of peers (“peer edges”).

At block 710, across all or a subset of individual 315 and or personality type matrix 330 records, relationship module 700 may determine peer relationship degree among individuals and organizations. For example, peer relationship degree may be determined based on and may comprise, for example, employment status, behavior 310 instances between the parties; for example, peer relationship degree may be based on shared intentions, goals, tasks, behaviors, and or outcomes; for example, peer relationship degree may be based on proximity of individuals in personality type matrix 330 records.

At block 715, relationship module 700 may determine potential peer relationships, recommendations for connection, for mentorship, and the like among individuals. Relationship module 700 may determine these potential relationships and recommendations for connection by identifying individuals with a similar peer relationship degree, of block 710, or who have or had a similar peer relationship degree but where one of the parties is older or further developed in employment status, and with whom an individual does not already communicate or with whom the individual does not have a mentor-mentee relationship. Relationship module 700 may determine these potential relationships and recommendations for connection by identifying peers with a similar peer relationship degree, of block 710, where one or more of the parties achieves or achieved better outcome(s) for similar intentions, goals, tasks, and or behaviors, where “better” may be based on a standard deviation. Parties identified as potential mentors may be offered tasks such as instructor, mentor, role model, and the like. An example of a mentorship recommendation may be seen in user interface 1100, illustrated in FIG. 11.

At block 720, relationship module 700 may identify peer harmonics within the nodes and edges, including synergistic, antagonistic, and neutral communication harmonics among peers.

FIG. 8 is a flow diagram illustrating an example of intention-behavior integration module 800 which may be performed by intention and behavior feedback computer device(s), such as intention and behavior feedback computer 200, according to some embodiments. These modules may be performed by or with the assistance of a hardware accelerator, such as hardware acceleration module 210.

At block 805, intention-behavior integration module 800 may train an artificial intelligence model, e.g. neural network, on intentions, goals, tasks, behaviors, peers, outcomes, and personality types matrices. An example of training is illustrated in block 806.

At block 810, intention-behavior integration module 800 may execute a neural network, e.g. a recurrent neural network or another neural network suitable to operate over time series data, and may provide the neural network with, for example, intention 305 records, goal 365 records, task 325 records, behavior 310 records, outcome 340 records, peer 360 records, and personality type matrix 330 records; in so doing, intention-behavior integration module 800 may encode the records as vectors, tensors, or the like and may match the size of the encoded records to the input size of the neural network.

At block 811, intention-behavior integration module 800 may train the neural network to predict behavior and or outcome based on intention 305, goal 365, task 325, peer 360, and personality type matrix 330 records.

At block 812, if not performed at block 812, intention-behavior integration module 800 may train the neural network to predict outcome and or behavior based on intention 305, goal 365, task 325, behavior 310, peer 360, and personality type matrix 330 records. Training on behavior and or outcome may or may not be performed in separate steps.

At Block 813, Intention-Behavior Integration Module 800 May Train the Neural Network to Predict Tasks Most Correlated with Outcomes, Based on Intention 305, Goal 365, and Personality Type Matrix 330 Records.

At block 815, intention-behavior integration module 800 may use a portion of the training data to compare predicted behaviors, outcomes, and tasks to actual to minimize a loss function, improve predictive ability of the artificial intelligence model and, for example, attention heads therein, through identification and weighting of more predictive input parameters. Blocks 810 and 815 may iterate until the prediction error is reduces to an acceptable level.

Opening loop block 820 to closing loop block 840 may iterate over individual 315 records.

At block 821, intention-behavior integration module 800 may train a local neural network, such as one executing on user interface computer device 111, on records for the then-current individual equivalent to the records of block 810 to block 815.

At block 825, intention-behavior integration module 800 may execute a neural network, e.g. a recurrent neural network or another neural network suitable to operate over time series data, to determine a predicted behavior and a predicted outcome of the predicted behavior with respect to the then-current individual based on, for example, the then-current individual's intentions, goals, tasks, peers, and personality type, e.g. with respect to intention 305, behavior 310, task 325, goal 365, peer 360, and personality type matrix 330 records for the individual.

At block 830, intention-behavior integration module 800 may execute the neural network and identify alternative behaviors, alternative outcomes, and alternative tasks with respect to the then-current individual. The alternative behaviors and alternative outcomes may be predicted behaviors and predicted outcomes with a lower probability of occurrence than those of block 825. The alternative tasks may be tasks more strongly correlated with a predicted or alternative outcome than a predicted task. One or more of the predicted behaviors, predicted outcomes, alternative predicted behaviors, alternative predicted outcomes, and or alternative tasks may be more or less desirable to the individual.

At block 835, intention-behavior integration module 800 may transmit output to user interface, such as user interface computer device 111, to identify the individual's personality type, e.g. in user interface 1000, current and predicted tasks, current and predicted behaviors, current and predicted outcomes, alternative predicted behaviors, alternative predicted outcomes, and alternative tasks to achieve the individual's intentions and goals, e.g. in user interface 1100.

At block 836, intention-behavior integration module 800 may transmit output to user interface 1100, such as user interface computer device 111, to suggest communication with others, such as mentors, such as mentors identified in block 715, who may have more experience, achieve preferred outcomes, or the like.

This output is based on neural network analysis of data from a large group of people. The neural network analysis effectively graphs, in a tractable manner, intentions, objectives, tasks, behaviors, and outcomes, in addition to personality types. The neural networks provide feedback to individuals, to allow them to see what they are doing, to predict what outcomes will come from these behaviors, to predict what behaviors they may do, to predict what may come from predicted behaviors, and to suggest alternative behaviors and tasks to achieve intentions and goas, in view of their personality type., for example, block 715 of relation module 700. These may be displayed to individuals as recommendations, with the ability of the individuals to act on the recommendations.

At block 899, intention-behavior integration module 800 may conclude and/or return to a module and/or another process which may have called it.

The Driver App, an example of which is illustrated in user interface 900 in FIG. 9, is an example of at least a portion of a user interface, as described herein, which may be used by an individual. The Driver App may comprise a series of interwoven, contextual tools. The Driver App is based on inputs which may be configured and contextualized for any industry. The inputs may comprise, for example, tasks, goals, and behaviors.

An individual using the Driver App works in order to achieve a goal; the goal may comprise an explicit, articulated component and or, as may often be case, may comprise an implicit, unconscious component.

Work may require the individual to perform a set of tasks. In order to achieve the task the individual employs behavior. Often, the behavior is habitual and is what the individual always does and which the individual thinks anyone would do in the same situation.

The individual may start out unconsciously believing or with the implicit belief that the behavior supports the task and that the tasks will help the individual achieve the individual's goals.

Through the individual's perception, the individual receives feedback regarding relationships, including with respect to peers, boss, employer, family. These relationships may be fragile. The individual may become aware that the individual is am not achieving the individual's stated goals and or that the individual's stated or articulated goals are not moving the individual towards personal freedom (the individual may state, “I am not any happier”).

The individual may enter into a state or process of reflection. The individual may start to realize that if the individual simply performs tasks using the individual's current behavior, the individual doesn't sustainably achieve the individual's goals (e.g. because the individual's goals aren't the individual's real or primary goals) and so the individual is not happier.

The individual may choose to adopt new behaviors by watching or inquiring as to what works for others, such as with feedback from the user interface. The individual may no longer have a firm belief that the individual's behavior will achieve the individual's goals. The result may be that the individual holds onto behaviors more loosely and may be a cautious time of self-assessment. Some of these, now questioned, behaviors may have been used during many good and bad times for the individual. The individual may be in a space where blame is an option and once the individual starts to question long-practiced behaviors, self-blame, recrimination etc., may be an issue.

The Driver App and user interface may suggest that the individual look at how others, particularly others with a similar personality type, act and may suggest relationships or other communication exchanges with others. If the individual can see patters, both similar and dissimilar, in others, the individual may understand that others have come through and grown through this. The individual may have a better chance of using this self-knowledge to become more healthy.

Gradually, alongside the individual's tasks, the individual sees that the individual's goals are separate from the individual and perhaps are not those of the individual at all. This may explain why, even if the goals are being routinely achieved, the individual does not necessarily feel more free or happy.

So as the individual did with the individual's behavior, now the individual starts to watch or inquire into what works for others and adapt goals and adopt behaviors alongside potentially even newer tasks, to achieve these new goals.

This relationship between tasks, goals and behaviors continues to evolve as the individual senses balance.

The individual share results with others (for example share CV) which creates further opportunities to achieve my goals

This may be illustrated in the user interface as movement from red to late stage orange, from concrete through subtle, from first perspective through 4th, from seeing everyone having the same reasoning to seeing myself as a collection of behaviors and objectives.

There is a stage where this dynamic may become inadequate. Either an individual cannot find the right behaviors to achieve the individual's goals OR the individual's goals don't capture what the individual really wants to achieve OR the individual sees that he/her cannot always achieve the other.

The individual may, with assistance, call on somebody to help, somebody who has achieved this goal or played with this behavior before. Someone who exhibits similar behavior in related tasks or goals.

The individual may not directly call on another individual but may ask or be prompted by the system. The system will look for patterns of goals, behaviors, and or tasks as the app records goals and behaviors in a personality profile, such as an enneagram type.

The Individual May See Patterns in how Peers Achieve Goals with the Peers' Behaviors. If an Individual Wants to Behave in a Different Way, it May be Helpful for the Individual to Learn about how Other Behave, Such as Those Who Achieved this Goal?

As these matches happen, the individual may start to reflect on the number of options there are and what worked for others. The options cause the individual to make decisions, including conscious decisions, and to take responsibility. This is a step towards moving past blame and becoming conscious.

Peers may share experiences. Individuals may decide whatever is most important in the context of the goal and/or behavior at hand.

This relationship between peers, tasks, goals, and behaviors may continue to evolve as the individual comes into harmony, balance, or greater self awareness and can begin to reliably achieve and reflect on goals, forming a feedback loop.

The user interface may bring in peers and unpeers.

There may be an overlapping stage in which the individual sees that the peer dynamic is adequate but not sufficient. The individual may reach goals, but they may not really be what the individual wants or needs because the individual is not moving towards freedom and happiness.

The individual may request to share details and see an overlay of the individual's differences relative to others.

The app may support the individual to track scenarios with perspective, to see triggers and trigger dissolution, and track progress toward modified and goals.

The user interface may map freedom—a relationship between metacapital reflection of the individual in 4 different perspectives (the individual, a group including the individual, a scenario and what drives it) allows behaviors to continue to become appropriate to each moment as the individual comes into harmony and balance and can begin to reliably choose goals that move toward freedom and behaviors that support them, forming a feedback loop

There may be a stage where it may become noticeable that all these dynamics offer only a step to the next.

An individual may call on a group that unbalances the individual (as before with an unpeer). The individual may deal with this through the user interface in the same way as for unpeers. Metacapital reflections regarding expectations and actual, including group expectations and actual and group reflection if available.

Groups or configuration can be family or work or other fragile relationships.

Instead of then going to behaviors or goals, the individual may reflect on shadows using meta capital. In addition, the individual may reflecting on ability to achieve balance (as balance or freedom/happiness may now be the goal) vs in other configurations, groups, or alone.

The app may support the individual to track balance (negotiation in meta capital).

Embodiments of the operations described herein may be implemented in a computer-readable storage device having stored thereon instructions that when executed by one or more processors perform the methods. The processor may include, for example, a processing unit and/or programmable circuitry. The storage device may include a machine readable storage device including any type of tangible, non-transitory storage device, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of storage devices suitable for storing electronic instructions. USB (Universal serial bus) may comply or be compatible with Universal Serial Bus Specification, Revision 2.0, published by the Universal Serial Bus organization, Apr. 27, 2000, and/or later versions of this specification, for example, Universal Serial Bus Specification, Revision 3.1, published Jul. 26, 2013. PCIe may comply or be compatible with PCI Express 3.0 Base specification, Revision 3.0, published by Peripheral Component Interconnect Special Interest Group (PCI-SIG), November 2010, and/or later and/or related versions of this specification.

As used in any embodiment herein, the term “logic” may refer to the logic of the instructions of an app, software, and/or firmware, and/or the logic embodied into a programmable circuitry by a configuration bit stream, to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

“Circuitry,” as used in any embodiment herein, may comprise, for example, singly or in any combination, hardwired circuitry, programmable circuitry such as FPGA. The logic may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc.

In some embodiments, a hardware description language (HDL) may be used to specify circuit and/or logic implementation(s) for the various logic and/or circuitry described herein. For example, in one embodiment the hardware description language may comply or be compatible with a very high speed integrated circuits (VHSIC) hardware description language (VHDL) that may enable semiconductor fabrication of one or more circuits and/or logic described herein. The VHDL may comply or be compatible with IEEE Standard 1076-1987, IEEE Standard 1076.2, IEEE1076.1, IEEE Draft 3.0 of VHDL-2006, IEEE Draft 4.0 of VHDL-2008 and/or other versions of the IEEE VHDL standards and/or other hardware description standards.

As used herein, the term “module” (or “logic”) may refer to, be part of, or include an Application Specific Integrated Circuit (ASIC), a System on a Chip (SoC), an electronic circuit, a programmed programmable circuit (such as, Field Programmable Gate Array (FPGA)), a processor (shared, dedicated, or group) and/or memory (shared, dedicated, or group) or in another computer hardware component or device that execute one or more software or firmware programs having executable machine instructions (generated from an assembler and/or a compiler) or a combination, a combinational logic circuit, and/or other suitable components with logic that provide the described functionality. Modules may be distinct and independent components integrated by sharing or passing data, or the modules may be subcomponents of a single module, or be split among several modules. The components may be processes running on, or implemented on, a single compute node or distributed among a plurality of compute nodes running in parallel, concurrently, sequentially or a combination, as described more fully in conjunction with the flow diagrams in the figures.

As used herein, a process corresponds to an instance of a program, e.g., an application program, executing on a processor and a thread corresponds to a portion of the process. A processor may include one or more execution core(s). The processor may be configured as one or more socket(s) that may each include one or more execution core(s).

Following are non-liming examples:

Example 1. An apparatus for intention and behavior feedback, comprising: a computer processor and a memory; and an intention and behavior feedback module to provide an individual with feedback regarding intentions and behaviors, wherein to provide the individual with feedback regarding intentions and behaviors, the intention and behavior feedback module is to obtain an intention or task of the individual, and with a neural network and based at least in part on the intention or task of the individual, determine a predicted behavior of the individual, determine a predicted outcome of the predicted behavior, and output the predicted outcome of the predicted behavior.

Example 2. The apparatus according to Example 1, wherein the neural network is trained on a database comprising a time series of intention, goal, task, behavior, outcome, and personality type matrix records for a plurality of individuals.

Example 3. The apparatus according to Example 2, wherein the neural network is further to graph at least one of intentions, goals, tasks, behaviors, and personality types relative to outcomes.

Example 4. The apparatus according to Example 2, wherein the neural network is further trained on a timer series of intention, goal, behavior, outcome, and personality type matrix records for the individual.

Example 5. The apparatus according to Example 1, wherein the neural network is further to determine an alternative behavior and an alternative predicted outcome of the alternative behavior and is further to output the alternative behavior and the alternative predicted outcome of the alternative behavior.

Example 6. The apparatus according to Example 1, wherein the neural network is further to determine an alternative task to achieve an intention or goal of the individual.

Example 7. The apparatus according to Example 1, wherein to obtain the intention or task of the individual, the intention and behavior feedback module is further to execute an intention module to obtain the intention or task of the individual, wherein to obtain the intention or task of the individual, the intention module is to obtain at least one of an organization associated with the individual, the intention of the individual, a task assigned to the individual, a behavior collection device associated with the individual and to be used to perform the task, and a response to a personality type question from the individual.

Example 8. The apparatus according to Example 1, wherein to provide the individual with feedback regarding intentions and behaviors, the intention and behavior feedback module is further to execute a behavior module to obtain a then-current behavior of the individual, wherein to obtain the then-current behavior of the individual, the behavior module is further to obtain the then-current behavior of the individual by monitoring a behavior collection device associated with the individual and is to match the then-current behavior of the individual with at least one of the intention of the individual, the task of the individual, or a goal of the individual.

Example 9. The apparatus according to Example 6, wherein to match the then-current behavior of the individual with at least one of the intention of the individual, the task of the individual, or the goal of the individual, the behavior module is to execute a behavior evaluation neural network.

Example 10. The apparatus according to Example 7, wherein the behavior module is further to execute the behavior evaluation neural network to interpret whether the then-current behavior is positive, negative, or neutral with respect to at least one of the intention of the individual, the task of the individual, or a goal of the individual.

Example 11. The apparatus according to Example 6, wherein the behavior collection device is used by the individual to perform the task.

Example 12. The apparatus according to Example 1, wherein the intention and behavior feedback module is further to obtain a response to a personality type question from the individual, determine a personality type matrix for the individual based on the response to the personality type question from the individual, and wherein to determine the predicted outcome of the predicted behavior further comprises to determine the predicted outcome of the predicted behavior informed by the personality type matrix for the individual, and wherein to determine the prediction regarding whether the behavior will block or aid the preferred outcome of the behavior comprises to determine the prediction regarding whether the behavior will block or aid the preferred outcome of the behavior relative to the personality type matrix for the individual.

Example 13. An computer implemented method for intention and behavior feedback, comprising: with a computer processor and a memory, providing an individual with feedback regarding intentions and behaviors, wherein providing the individual with feedback regarding intentions and behaviors comprises obtaining an intention or task of the individual, performing neural network and based at least in part on the intention or task of the individual, determining a predicted behavior of the individual, determining a predicted outcome of the predicted behavior, and outputting the predicted outcome of the predicted behavior.

Example 14. The method according to Example 13, further comprising training the neural network is trained on a database comprising a time series of intention, goal, task, behavior, outcome, and personality type matrix records for a plurality of individuals.

Example 15. The method according to Example 14, further comprising the neural network graphing at least one of intentions, goals, tasks, behaviors, and personality types relative to outcomes.

Example 16. The method according to Example 14, further comprising training the neural network on a timer series of intention, goal, behavior, outcome, and personality type matrix records for the individual.

Example 17. The method according to Example 13, further comprising the neural network determining an alternative behavior and an alternative predicted outcome of the alternative behavior and further outputting the alternative behavior and the alternative predicted outcome of the alternative behavior.

Example 18. The method according to Example 13, further comprising the neural network determining an alternative task to achieve an intention or goal of the individual.

Example 19. The method according to Example 13, wherein obtaining the intention or task of the individual further comprises obtaining at least one of an organization associated with the individual, the intention of the individual, a task assigned to the individual, a behavior collection device associated with the individual and to be used to perform the task, and a response to a personality type question from the individual.

Example 20. The method according to Example 13, wherein providing the individual with feedback regarding intentions and behaviors further comprises obtaining a then-current behavior of the individual, wherein obtaining the then-current behavior of the individual comprising obtaining the then-current behavior of the individual by monitoring a behavior collection device associated with the individual and matching the then-current behavior of the individual with at least one of the intention of the individual, the task of the individual, or a goal of the individual.

Example 21. The method according to Example 20, further comprising matching the then-current behavior of the individual with at least one of the intention of the individual, the task of the individual, or the goal of the individual, the behavior module by executing a behavior evaluation neural network.

Example 22. The method according to Example 21, further comprising the behavior evaluation neural network interpreting whether the then-current behavior is positive, negative, or neutral with respect to at least one of the intention of the individual, the task of the individual, or a goal of the individual.

Example 23. The method according to Example 20, wherein the behavior collection device is used by the individual to perform the task.

Example 24. The method according to Example 13, further comprising obtaining a response to a personality type question from the individual, determining a personality type matrix for the individual based on the response to the personality type question from the individual, and wherein determining the predicted outcome of the predicted behavior further comprises determining the predicted outcome of the predicted behavior informed by the personality type matrix for the individual, and wherein determining the prediction regarding whether the behavior will block or aid the preferred outcome of the behavior comprises determining the prediction regarding whether the behavior will block or aid the preferred outcome of the behavior relative to the personality type matrix for the individual.

Example 25. A computer apparatus for intention and behavior feedback, comprising: means to provide an individual with feedback regarding intentions and behaviors, wherein means to provide the individual with feedback regarding intentions and behaviors comprises means to obtain an intention or task of the individual, means to perform a neural network and, based at least in part on the intention or task of the individual, means for the neural network to determine a predicted behavior of the individual, determine a predicted outcome of the predicted behavior, and output the predicted outcome of the predicted behavior.

Example 26. The computer apparatus according to Example 25, further comprising means to train the neural network on a database comprising a time series of intention, goal, task, behavior, outcome, and personality type matrix records for a plurality of individuals.

Example 27. The computer apparatus according to Example 26, further comprising the neural network graphing at least one of intentions, goals, tasks, behaviors, and personality types relative to outcomes.

Example 28. The computer apparatus according to Example 26, further comprising means to train the neural network on a timer series of intention, goal, behavior, outcome, and personality type matrix records for the individual.

Example 29. The computer apparatus according to Example 25, further comprising means for the neural network to determine an alternative behavior and an alternative predicted outcome of the alternative behavior and to further output the alternative behavior and the alternative predicted outcome of the alternative behavior.

Example 30. The computer apparatus according to Example 25, further comprising means for the neural network to determine an alternative task to achieve an intention or goal of the individual.

Example 31. The computer apparatus according to Example 25, wherein means to obtain the intention or task of the individual further comprises means to obtain at least one of an organization associated with the individual, the intention of the individual, a task assigned to the individual, a behavior collection device associated with the individual and to be used to perform the task, and a response to a personality type question from the individual.

Example 32. The computer apparatus according to Example 25, wherein means to provide the individual with feedback regarding intentions and behaviors further comprises means to obtain a then-current behavior of the individual, wherein means to obtain the then-current behavior of the individual comprises means to obtain the then-current behavior of the individual by monitoring a behavior collection device associated with the individual and means to match the then-current behavior of the individual with at least one of the intention of the individual, the task of the individual, or a goal of the individual.

Example 33. The computer apparatus according to Example 32, further comprising means to match the then-current behavior of the individual with at least one of the intention of the individual, the task of the individual, or the goal of the individual, the behavior module with a behavior evaluation neural network.

Example 34. The computer apparatus according to Example 33, further comprising means for the behavior evaluation neural network to interpret whether the then-current behavior is positive, negative, or neutral with respect to at least one of the intention of the individual, the task of the individual, or a goal of the individual.

Example 35. The computer apparatus according to Example 32, wherein the behavior collection device is used by the individual to perform the task.

Example 36. The computer apparatus according to Example 25, further comprising means to obtain a response to a personality type question from the individual, determine a personality type matrix for the individual based on the response to the personality type question from the individual, and wherein means to determine the predicted outcome of the predicted behavior further comprises means to determine the predicted outcome of the predicted behavior informed by the personality type matrix for the individual, and wherein means to determine the prediction regarding whether the behavior will block or aid the preferred outcome of the behavior comprises means to determine the prediction regarding whether the behavior will block or aid the preferred outcome of the behavior relative to the personality type matrix for the individual.

Example 37. One or more computer-readable media comprising instructions that cause a computer device, in response to execution of the instructions by a processor of the computer device, to: provide an individual with feedback regarding intentions and behaviors, wherein means to provide the individual with feedback regarding intentions and behaviors comprises to obtain an intention or task of the individual, perform a neural network and, based at least in part on the intention or task of the individual, determine a predicted behavior of the individual, determine a predicted outcome of the predicted behavior, and output the predicted outcome of the predicted behavior.

Example 38. The computer-readable media according to Example 37, wherein the instructions further cause the computer device to train the neural network on a database comprising a time series of intention, goal, task, behavior, outcome, and personality type matrix records for a plurality of individuals.

Example 39. The computer-readable media according to Example 38, wherein the instructions further cause the neural network to graph at least one of intentions, goals, tasks, behaviors, and personality types relative to outcomes.

Example 40. The computer-readable media according to Example 38, wherein the instructions further cause the computer device to train the neural network on a timer series of intention, goal, behavior, outcome, and personality type matrix records for the individual.

Example 41. The computer-readable media according to Example 37, wherein the instructions further cause the neural network to determine an alternative behavior and an alternative predicted outcome of the alternative behavior and to further output the alternative behavior and the alternative predicted outcome of the alternative behavior.

Example 42. The computer-readable media according to Example 37, wherein the instructions further cause the neural network to determine an alternative task to achieve an intention or goal of the individual.

Example 43. The computer-readable media according to Example 37, wherein to obtain the intention or task of the individual further comprises to obtain at least one of an organization associated with the individual, the intention of the individual, a task assigned to the individual, a behavior collection device associated with the individual and to be used to perform the task, and a response to a personality type question from the individual.

Example 44. The computer-readable media according to Example 37, to provide the individual with feedback regarding intentions and behaviors further comprises to obtain a then-current behavior of the individual, wherein to obtain the then-current behavior of the individual comprises to obtain the then-current behavior of the individual by monitoring a behavior collection device associated with the individual and wherein the instructions further cause the computer device to match the then-current behavior of the individual with at least one of the intention of the individual, the task of the individual, or a goal of the individual.

Example 45. The computer-readable media according to Example 44, wherein the instructions further cause the computer device to match the then-current behavior of the individual with at least one of the intention of the individual, the task of the individual, or the goal of the individual, the behavior module with a behavior evaluation neural network.

Example 46. The computer-readable media according to Example 45, wherein the instructions further cause the behavior evaluation neural network to interpret whether the then-current behavior is positive, negative, or neutral with respect to at least one of the intention of the individual, the task of the individual, or a goal of the individual.

Example 47. The computer-readable media according to Example 44, wherein the behavior collection device is used by the individual to perform the task.

Example 48. The computer-readable media according to Example 37, wherein the instructions further cause the computer device to obtain a response to a personality type question from the individual, determine a personality type matrix for the individual based on the response to the personality type question from the individual, and wherein to determine the predicted outcome of the predicted behavior further comprises to determine the predicted outcome of the predicted behavior informed by the personality type matrix for the individual, and wherein to determine the prediction regarding whether the behavior will block or aid the preferred outcome of the behavior comprises to determine the prediction regarding whether the behavior will block or aid the preferred outcome of the behavior relative to the personality type matrix for the individual.

1. An apparatus for intention and behavior feedback, comprising:
a computer processor and a memory; and
an intention and behavior feedback module in the memory to provide an individual with feedback regarding intentions and behaviors, wherein to provide the individual with feedback regarding intentions and behaviors, the intention and behavior feedback module is to obtain an intention or task of the individual, and with a neural network and based at least in part on the intention or task of the individual, determine a predicted behavior of the individual, determine a predicted outcome of the predicted behavior, and output the predicted outcome of the predicted behavior.

2. The apparatus according to claim 1, wherein the neural network is further to determine an alternative behavior and an alternative predicted outcome of the alternative behavior and is further to output the alternative behavior and the alternative predicted outcome of the alternative behavior.

3. The apparatus according to claim 1, wherein the neural network is further to determine an alternative task to achieve an intention or goal of the individual.

4. The apparatus according to claim 1, wherein to obtain the intention or task of the individual, the intention and behavior feedback module is further to execute an intention module to obtain the intention or task of the individual, wherein to obtain the intention or task of the individual, the intention module is to obtain at least one of an organization associated with the individual, the intention of the individual, a task assigned to the individual, a behavior collection device associated with the individual and to be used to perform the task, and a response to a personality type question from the individual.

5. The apparatus according to claim 1, wherein to provide the individual with feedback regarding intentions and behaviors, the intention and behavior feedback module is further to execute a behavior module to obtain a then-current behavior of the individual, wherein to obtain the then-current behavior of the individual, the behavior module is further to obtain the then-current behavior of the individual by monitoring a behavior collection device associated with the individual and is to match the then-current behavior of the individual with at least one of the intention of the individual, the task of the individual, or a goal of the individual.

6. The apparatus according to claim 5, wherein to match the then-current behavior of the individual with at least one of the intention of the individual, the task of the individual, or the goal of the individual, the behavior module is to execute a behavior evaluation neural network, wherein the behavior module is further to execute the behavior evaluation neural network to interpret whether the then-current behavior is positive, negative, or neutral with respect to at least one of the intention of the individual, the task of the individual, or a goal of the individual.

7. A computer implemented method for intention and behavior feedback, comprising:
with a computer processor and a memory, providing an individual with feedback regarding intentions and behaviors, wherein providing the individual with feedback regarding intentions and behaviors comprises obtaining an intention or task of the individual, performing neural network and based at least in part on the intention or task of the individual, determining a predicted behavior of the individual, determining a predicted outcome of the predicted behavior, and outputting the predicted outcome of the predicted behavior.

8. The method according to claim 7, further comprising training the neural network is trained on a database comprising a time series of intention, goal, task, behavior, outcome, and personality type matrix records for a plurality of individuals.

9. The method according to claim 8, further comprising training the neural network on a timer series of intention, goal, behavior, outcome, and personality type matrix records for the individual.

10. The method according to claim 7, further comprising the neural network determining an alternative behavior and an alternative predicted outcome of the alternative behavior and further outputting the alternative behavior and the alternative predicted outcome of the alternative behavior and further comprising the neural network determining an alternative task to achieve an intention or goal of the individual.

11. The method according to claim 7, wherein obtaining the intention or task of the individual further comprises obtaining at least one of an organization associated with the individual, the intention of the individual, a task assigned to the individual, a behavior collection device associated with the individual and to be used to perform the task, and a response to a personality type question from the individual.

12. The method according to claim 7, wherein providing the individual with feedback regarding intentions and behaviors further comprises obtaining a then-current behavior of the individual, wherein obtaining the then-current behavior of the individual comprising obtaining the then-current behavior of the individual by monitoring a behavior collection device associated with the individual and matching the then-current behavior of the individual with at least one of the intention of the individual, the task of the individual, or a goal of the individual and further comprising matching the then-current behavior of the individual with at least one of the intention of the individual, the task of the individual, or the goal of the individual, the behavior module by executing a behavior evaluation neural network.

13. The method according to claim 12, further comprising the behavior evaluation neural network interpreting whether the then-current behavior is positive, negative, or neutral with respect to at least one of the intention of the individual, the task of the individual, or a goal of the individual.

14. The method according to claim 7, further comprising obtaining a response to a personality type question from the individual, determining a personality type matrix for the individual based on the response to the personality type question from the individual, and wherein determining the predicted outcome of the predicted behavior further comprises determining the predicted outcome of the predicted behavior informed by the personality type matrix for the individual, and wherein determining the prediction regarding whether the behavior will block or aid the preferred outcome of the behavior comprises determining the prediction regarding whether the behavior will block or aid the preferred outcome of the behavior relative to the personality type matrix for the individual.

15. One or more computer-readable media comprising instructions that cause a computer device, in response to execution of the instructions by a processor of the computer device, to:
provide an individual with feedback regarding intentions and behaviors, wherein means to provide the individual with feedback regarding intentions and behaviors comprises to obtain an intention or task of the individual, perform a neural network and, based at least in part on the intention or task of the individual, determine a predicted behavior of the individual, determine a predicted outcome of the predicted behavior, and output the predicted outcome of the predicted behavior.

16. The computer-readable media according to claim 15, wherein the instructions further cause the computer device to train the neural network on a database comprising a time series of intention, goal, task, behavior, outcome, and personality type matrix records for a plurality of individuals and wherein the instructions further cause the computer device to train the neural network on a timer series of intention, goal, behavior, outcome, and personality type matrix records for the individual.

17. The computer-readable media according to claim 15, wherein the instructions further cause the neural network to determine an alternative behavior and an alternative predicted outcome of the alternative behavior and to further output the alternative behavior and the alternative predicted outcome of the alternative behavior and wherein the instructions further cause the neural network to determine an alternative task to achieve an intention or goal of the individual.

18. The computer-readable media according to claim 15, wherein to obtain the intention or task of the individual further comprises to obtain at least one of an organization associated with the individual, the intention of the individual, a task assigned to the individual, a behavior collection device associated with the individual and to be used to perform the task, and a response to a personality type question from the individual.

19. The computer-readable media according to claim 15, to provide the individual with feedback regarding intentions and behaviors further comprises to obtain a then-current behavior of the individual, wherein to obtain the then-current behavior of the individual comprises to obtain the then-current behavior of the individual by monitoring a behavior collection device associated with the individual and wherein the instructions further cause the computer device to match the then-current behavior of the individual with at least one of the intention of the individual, the task of the individual, or a goal of the individual.

20. The computer-readable media according to claim 15, wherein the instructions further cause the computer device to obtain a response to a personality type question from the individual, determine a personality type matrix for the individual based on the response to the personality type question from the individual, and wherein to determine the predicted outcome of the predicted behavior further comprises to determine the predicted outcome of the predicted behavior informed by the personality type matrix for the individual, and wherein to determine the prediction regarding whether the behavior will block or aid the preferred outcome of the behavior comprises to determine the prediction regarding whether the behavior will block or aid the preferred outcome of the behavior relative to the personality type matrix for the individual.
